(19) United States
(12) Patent Application Publication
(10) Pub. No.: US 2013/0035559 A1
(43) Pub. Date: Feb. 7, 2013

(54) STIMULATION OF SATIETY HORMONE RELEASE

(71) Applicants: Pamela J. Hornby, Radnor, PA (US); Tatiana Ort, Radnor, PA (US); Radhika Kajekar, Radnor, PA (US); Paul R. Wade, Philadelphia, PA (US)

(72) Inventors: Pamela J. Hornby, Radnor, PA (US); Tatiana Ort, Radnor, PA (US); Radhika Kajekar, Radnor, PA (US); Paul R. Wade, Philadelphia, PA (US)

(21) Appl. No.: 13/464,470

(22) Filed: May 4, 2012

Related U.S. Application Data
(63) Continuation of application No. 12/546,721, filed on Aug. 25, 2009, now abandoned.
(60) Provisional application No. 61/091,748, filed on Aug. 26, 2008.

(51) Int. Cl. A61M 37/00; A61B 5/145; A61B 5/05

(52) U.S. Cl. 600/301; 604/500; 600/554

(57) ABSTRACT

The present invention provides a site specific way to enhance a natural hormonal response to nutrients entering the small intestine after gastric emptying, thereby providing therapeutic value for obesity or diabetic patients. In one aspect, the present invention provides methods of stimulating the release of satiety hormone in a subject comprising applying a first electrical stimulus to a tissue in the lumen of the gastrointestinal system of the subject contemporaneously with the contacting of L-cells of the tissue with a nutrient stimulus. The present invention also provides methods for predicting patient response to a weight loss surgery comprising applying a first electrical stimulus to a tissue of the gastrointestinal system of said patient contemporaneously with the contacting of L-cells of the tissue with a nutrient stimulus, assessing the effect of the electrical stimulus in said patient, and, correlating said effect to said patient's response to a weight loss surgery.

### CROSS-REFERENCE TO RELATED APPLICATIONS

This application claims the priority of U.S. Provisional

Application Ser. No. 61/091,748, filed 26 Aug. 2008, the entire contents of which is incorporated herein by reference in its entirety.

### FIELD OF THE INVENTION

The present invention relates generally to diagnosis and/or treatment of metabolic disorders using electrical stimulation.

### BACKGROUND OF THE INVENTION

Humans have evolved to conserve energy in times of food scarcity. With food readily available to most in the western world, the ability to store excess energy has contributed to the increased frequency of morbidly obese patients and those with Type 2 Diabetes (T2D). Together, the diseases of obesity and T2D affect about 80 million people in the U.S. and about 500 million people worldwide. Patients having such conditions have increased morbidity and mortality resulting from associated co-morbidities, including cardiovascular disease and arthritis.

A new class of drugs that are similar to a key hormone that regulates the body's own glucose control hormone, Glucagon-Like Peptide (GLP-1), has led to some advances in the attempt to alleviate T2D and obesity and have been termed “incretin mimetics.” Exenatide is an incretin mimetic that improves both glucose control and weight loss (Schnabel C A, Wintle M, and Kolterman O. Metabolic effects of the incretin mimetic exenatide in the treatment of type 2 diabetes. Vasc Health Risk Manag 2: 69-77, 2006). Normally, the presence of nutrients, which arise from a meal consisting of carbohydrates, fats and proteins, termed ‘digesta’ in the digestive tract, stimulates release of the body's own incretins into the blood stream. Key hormones, released by specialized L-cells located in the mucosa, which is the innermost interior (luminal) wall of the intestines, coordinate the body's response to a meal. The hormones produce this effect by inducing a sense of fullness and cessation of eating (satiety), triggering the release of insulin to maintain proper glucose levels (incretin effect) and slowing the passage of contents through the digestive tract (delaying gastric emptying and slowing small intestinal transit). Collectively, these effects have been termed the ileal brake.

The term ileal brake, when originally coined in 1984 by Spiller, referred to the action of PeptideYY (Spiller R C, Trotman I F, Higgins B E, Ghatei M A, Grimble G K, Lee Y C, Bloom S R, Misiewicz J J, and Silk D B. The ileal brake—inhibition of jejunal motility after ileal fat perfusion in man. Gut 25: 365-374, 1984); however, recent research has expanded the understanding of the complexity of this important mechanism, both in terms of the hormones that play a role (such as PYY, GLP-1, and GLP-2, among others), as well as the multiplicity of effects of release of those hormones (gastric emptying, a feeling of fullness cessation of eating, triggering of insulin secretion)

An insufficient ileal brake, i.e., the inability of the body to release sufficient quantities of these hormones in response to a meal, is a contributory factor in obesity and T2D. In non-obese non-diabetic individuals fasting levels of GLP-1 are in the range of 5-10 pmol/L and increase rapidly to 15-50 pmol/L after a meal (Drucker D J, and Nauck M A. The incretin system: glucagon-like peptide-1 receptor agonists and dipeptidyl peptidase-4 inhibitors in type 2 diabetes. Lancet 368: 1696-1705, 2006). In T2D patients, the meal-related increase in GLP-1 is significantly blunted (Toft-Nielsen M B, Damholt M B, Madsbad S, Hilsted L M, Hughes T E, Michelsen B K, and Holst J J Determinants of the impaired secretion of glucagon-like peptide-1 in type 2 diabetic patients. J Clin Endocrinol Metab 86: 3717-3723, 2001). The decreased insulin levels of such patients are attributable to an insufficient level of GLP-1, not an inadequate pancreatic response to GLP-2 to release insulin (Toft-Nielsen M B, Madsbad S, and Holst J J. Continuous subcutaneous infusion of glucagon-like peptide 1 lowers plasma glucose and reduces appetite in type 2 diabetic patients. Diabetes Care 22: 1137-1143, 1999). Similarly, obese subjects have lower basal fasting hormone levels and have a smaller meal-associated rise (Small C J, and Bloom S R. Gut hormones and the control of appetite. Trends Endocrinol Metab 15: 259-263, 2004). Therefore, enhancing the body's endogenous levels of GLP-1 would be expected to have impact on both obesity and diabetes.

GLP-1 exists in several forms. Within the cell, the precursor of GLP-1 is proglucagon, which is cleaved to form GLP-1-(1-37), then the next step is the removal of the first six amino acids from the N terminus to form the two known biologically active forms of GLP-1. A majority of GLP-1 (−80%) is amidated to form GLP-1 (7-36)NH₂, and a minority (˜20%) is GLP-1-(7-37). This proteolytic processing occurs within the cell and before secretion and these two forms comprise the biologically active forms of GLP-1. Both GLP-1-(7-36)NH₂ and GLP-1-(7-37) increase the insulin response to glucose, then, after release, GLP-1 is metabolized by the protease dipeptidyl peptidase IV (DPP-IV) into GLP-1-(9-36) amide, which is inactive humans (Vahl T P, Paty B W, Fuller B D, Prigeon R L, and D'Alessio D A. Effects of GLP-1-(7-36)NH2, GLP-1-(7-37), and GLP-1-(9-36)NH2 on intravenous glucose tolerance and glucose-induced insulin secretion in healthy humans. J Clin Endocrinol Metab 88: 1772-1779, 2003). Pharmaceutical means to increasing the endogenous active forms of GLP-1 include inhibition of its breakdown by dipeptidyl peptidase-4 (DPP-4) inhibitors, such as vildagliptin. In diabetic patients, improvement in glucose control is obtained by increasing the circulating levels of GLP-1 by vildagliptin (Ahren B, Pacini G, Foley J E, and Schweizer A. Improved meal-related beta-cell function and insulin sensitivity by the dipeptidyl peptidase-IV inhibitor vildagliptin in metformin-treated patients with type 2 diabetes over 1 year. Diabetes Care 28: 1936-1940, 2005).

There exists an unmet need for treatment among T2D and obesity patients who are not well managed by pharmacological treatments alone. Currently, the most effective treatment for morbid obesity is bariatric surgery, which improves weight loss and T2D in 77% of patients with co-morbidity (Buchwald H, Avidor Y, Braunwald E, Jensen M D, Pories W, Fahrbach K, and Schoelles K Bariatric surgery: a systematic review and meta-analysis. Jama 292: 1724-1737, 2004). After Roux-en-Y gastric bypass surgery in morbidly obese patients hormone levels change even before significant weight loss occurs (Rubino F, Gagner M Gentileschi P, Kini S, Fukuyama S, Feng J, and Diamond E. The early effect of the Roux-en-Y gastric bypass on hormones involved in body weight regulation and glucose metabolism. Ann Surg 240: 236-242, 2004). A number of studies in patients after bariatric surgery suggest that the incretin pathway contributes to the improvements in T2D and weight loss noted. Specifically, there are increases in meal-related circulating GLP-1 levels after surgery (Laferrere B, Heshka S, Wang K, Khan Y, McGinty J, Teixeira J, Hart A B, and Olivan B. Incretin levels and effect are markedly enhanced 1 month after Roux-en-Y gastric bypass surgery in obese patients with type 2 diabetes. Diabetes Care 30: 1709-1716, 2007; Whitson B A, Leslie D B, Kellogg T A, Maddaus M A, Buchwald H, Billington C J, and Ikramuddin S. Entero-endocrine changes after gastric bypass in diabetic and nondiabetic patients: a preliminary study. J Surg Res 141: 31-39, 2007). However, bariatric surgery is perceived as an extreme measure and is currently recommended only for morbidly obese patients. At the 2008 American Diabetes Association meeting, Dr. C. H. Sorli, M.D. (Billings Clinic, Montana) reported a less invasive approach using an investigational bypass that included an impermeable fluoropolymer sleeve placed via an endoscope and fastened with a barbed metal anchor at the duodenal entrance. This sleeve improved glucose control for one week in 16 patients although over the short time of the study weight loss was not observed.

Thus, there would be advantages over invasive bariatric surgery for a device that improved both weight loss and glucose control with the prospect of a shorter procedure, without general anesthesia, and that is easily reversible.

### SUMMARY OF THE INVENTION

In one aspect, the present invention provides methods of stimulating the release of satiety hormone(s) in a subject comprising applying a first electrical stimulus to a tissue in the gastrointestinal system of the subject contemporaneously with the contacting of L-cells of the tissue with a nutrient stimulus. In another aspect, the present invention provides methods for predicting patient response to a weight loss surgery comprising applying a first electrical stimulus to a tissue of the gastrointestinal system of said patient contemporaneously with the contacting of L-cells of the tissue with a nutrient stimulus, assessing the effect of the electrical stimulus in said patient, and, correlating said effect to said patient's response to a weight loss surgery.

### BRIEF DESCRIPTION OF THE DRAWINGS

FIG. 1 depicts an assembly used to apply electric stimulus to dissected rat ileum.

FIG. 2 shows the concentration of GLP-1 in segments from the entire GI tract released after 45 minutes incubation in linoleic acid.

FIG. 3 depicts the results of an analysis of epithelial mucosa from the small and large intestine for the presence GLP-1.

FIG. 4 shows the increase of GLP-1 concentration over time during incubation in Krebs Ringers bicarbonate buffer with (two examples) and without 3 mg/mL linoleic acid.

FIG. 5 provides a plot of the difference in GLP-1 released in response to various electrical stimulation conditions in the presence of linoleic acid as compared with paired samples exposed to linoleic acid alone.

FIG. 6 presents the same data as the preceding figure as a percentage of GLP-1 released in response to various electrical stimulation conditions.

FIG. 7 illustrates effect of a neurotoxin on the effect of linoleic acid-induced release of GLP-1, with and without electric stimulation.

FIG. 8 shows that the average charge (Qₐᵥₑ) delivered per phase during stimulation is a function of the average current (Iₐᵥₑ) and pulse width (PW).

FIG. 9 depicts the change in muscle tone of isolated ileum after 40 minutes under various incubation and stimulation conditions.

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

In the present disclosure the singular forms “a,” “an,” and “the” include the plural reference, and reference to a particular numerical value includes at least that particular value, unless the context clearly indicates otherwise. Thus, for example, a reference to “a stimulus” is a reference to one or more of such stimuli and equivalents thereof known to those skilled in the art, and so forth. When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” refers to a value of 7.2 to 8.8, inclusive; as another example, the phrase “about 8%” refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like.

The disclosures of each patent, patent application, and publication cited or described in this document are hereby incorporated herein by reference, in their entirety.

The present invention provides, among other things, a site specific way to enhance the body's endogenous GLP-1 response to nutrients entering the small intestine, thereby providing therapeutic value for obesity or diabetic patients. As described herein, it has been discovered that specific regimes of electrical stimulation of the intestine enhance the release of a principal satiety hormone. As shown herein, electrical stimulation can be applied to a segment of isolated intestine to enhance GLP-1 release in response to a nutrient, linoleic acid. Furthermore, it is demonstrated that electrical stimulation can act directly on the cells in the gut that produce these hormones in response to nutrient: the L-cells. L-cells release ileal brake hormones that modulate insulin secretion, glucose homeostasis, gastric emptying, intestinal transit, and a feeling of fullness. They are located throughout the small and large intestines with the greatest numbers of cells located in the distal small intestine (ileum) and the proximal colon. Interestingly, in T2D the number of L cells in the intestine is increased (Theodorakis M J, Carlson O, Michopoulos S, Doyle M E, Juhaszova M, Petraki K, and Egan J M. Human duodenal enteroendocrine cells: source of both incretin peptides, GLP-1 and GIP. Am J Physiol Endocrinol Metab 290: E550-559, 2006), as if the body is trying to compensate for the blunted release of hormones in these patients.

An advantage of using site-selective electrical stimulation to enhance the intestinal release of GLP-1, as disclosed herein, is that the increased GLP-1 acts locally within a few minutes of release on GLP-1. A local site of action of GLP-1 is on its own receptors on the vagus nerve endings that are present in the intestinal and hepatic portal vascular circulation (Vahl T P, Tauchi M, Durler T S, Elfers E E, Fernandes T M, Bitner R D, Ellis K S, Woods S C, Seeley R J, Herman J P, and D'Alessio D A. GLP-1 receptors expressed on nerve terminals in the portal vein mediate the effects of endogenous GLP-1 on glucose tolerance in rats. Endocrinology 2007). Thus the increased GLP-1 released produces its effects locally while normal breakdown of the circulating GLP-1 is not inhibited. This approach would be expected to have fewer adverse effects than administration of exogenous pharmacological agents. Thus, the electrical stimulation within the intestines can be employed in order to allow the body to do what it naturally does, when it naturally does it, but in a more effective way.

Electrical stimulation devices implanted in the stomach of obese patients have been reported to have variable positive effects on weight loss (Zhang C, Ng K L, Li J D, He F, Anderson D J, Sun Y E, and Zhou Q Y. Prokineticin 2 is a target gene of proneural basic helix-loop-helix factors for olfactory bulb neurogenesis. J Biol Chem 282: 6917-6921, 2007), with improvements in glucose control in T2D patients secondary to weight loss. This stimulation would not be expected to act directly on L-cells since these cells are absent from the stomach. Intestinal electrical stimulation studies in obese or diabetic patients are fewer in number and tend to report the resulting neural and motility effects. For example, in diabetic neuropathy, electrical stimulation of the duodenum, which is located at the oral end of the small intestine, results in nerve responses that are weaker than in control patients (Frokjaer J B, Andersen S D, Ejskaer N, Funch-Jensen P, Arendt-Nielsen L, Gregersen H, and Drewes A M Gut sensations in diabetic autonomic neuropathy. Pain 131: 320-329, 2007). In healthy volunteers duodenal electrical stimulation delays gastric emptying and reduces water intake (Liu S, Hou X, and Chen J D. Therapeutic potential of duodenal electrical stimulation for obesity: acute effects on gastric emptying and water intake. Am J Gastroenterol 100: 792-796, 2005). In preclinical models in rat and dog, stimulation of the duodenum at the proximal (oral) end of the small intestine (20 Hz, 6 mA, 300 ms) reduces food intake and this effect is sustained over 4 weeks stimulation in rats (Yin J, Ouyang H, and Chen J D. Potential of intestinal electrical stimulation for obesity: a preliminary canine study. Obesity (Silver Spring) 15: 1133-1138, 2007; Yin J, Zhang J, and Chen J D. Inhibitory effects of intestinal electrical stimulation on food intake, weight loss and gastric emptying in rats. Am J Physiol Regul Integr Comp Physiol 293: R78-82, 2007). The positive effect on food intake is ascribed to motility changes in these studies and not attributed to altered hormone levels, which were not reported.

Altering the activity of nerves, such as the vagus and sympathetic nerves, by electrical stimulation can modulate GLP-1, although direct electrical stimulation of the vagus nerve supplying the pig ileum has been shown to have only a weak stimulatory effect on GLP-1 release (Hansen L, Lampert S, Mineo H, and Holst J J Neural regulation of glucagon-like peptide-1 secretion in pigs. Am J Physiol Endocrinol Metab 287: E939-947, 2004). It is well known that the vagus nerve senses food entering the stomach and, by long reflex loops coordinates this information via the brain and back down to the intestine to prepare the intestine for an ileal brake response by inducing an increase in GLP-1 (Rocca A S, and Brubaker P L. Role of the vagus nerve in mediating proximal nutrient-induced glucagon-like peptide-1 secretion. Endocrinology 140: 1687-1694, 1999). In U.S. Pub. No. 2007/0179556, an experiment is described wherein electrical stimulation applied by a device surgically implanted on the distal ileum of dog resulted in alterations in the timing of release and blood levels of GLP-1. The reflex mechanisms are mimicked by surgical insertion of an electrical impedance sensing device implanted in the stomach to determine the stomach's cross-sectional area, combined with an electrical stimulation device implanted in the intestines to cause GLP-1 release (Id.). The increase in cross-sectional area of the stomach is associated with changes in gastric motility and satiation.

It has presently been discovered that electrical stimulation parameters used alone do not cause release of GLP-1 from intestinal L-cells, unless such cells are concurrently exposed to a nutrient, such as linoleic acid, which is known to normally release ileal brake hormones. This finding indicates that a locally implanted intestinal electrical stimulation device can be designed to be temporally effective, because it would enhance GLP-1 release only when stimulation was partially contemporaneous with a nutrient stimulus.

The second unexpected result demonstrated herein is that electrical stimulation enhances GLP-1 release in response to linoleic acid in the presence of a neurotoxin. The presence of tetrodotoxin at a concentration (0.5 μM) that prevents nerve communication by blocking sodium channels did not prevent a two-fold increase in GLP-1 evoked by direct electrical stimulation of ileal tissue. L-cells are not derived from the same embryological lineage as neuronal cells, however they share many of the characteristics of nerve cells. Neuronal type ion channels (Reimann F, Maziarz M, Flock G, Habib A M, Drucker D J, and Gribble F M Characterization and functional role of voltage gated cation conductances in the glucagon-like peptide-1 secreting GLUTag cell line. J Physiol 563: 161-175, 2005; Gameiro A, Reimann F, Habib AM, O'Malley D, Williams L, Simpson A K, and Gribble F M. The neurotransmitters glycine and GABA stimulate glucagon-like peptide-1 release from the GLUTag cell line. J Physiol 569: 761-772, 2005) have been identified on a GLP-1 secreting intestinal cell line. While not intending to be bound by any particular theory, it can be envisioned that electrical stimulation directly alters the excitability of the cells in the intestine in situ and increase their hormonal response to a nutrient stimulus.

Thus, electrical stimulation of the small intestine favorably changes the release of at least one, and possibly a suite, of hormones from endocrine cells (including, for example, L-cells) directly, independent of nerve stimulation, in response to a nutrient luminal stimulus. Essentially, the precise manner of electrical stimulation disclosed herein creates a power-assisted ileal brake.

In one aspect, the present invention provides methods of stimulating the release of satiety hormone in a subject comprising applying a first electrical stimulus to a tissue of the gastrointestinal system of the subject contemporaneously with the contacting of L-cells of the tissue with a nutrient stimulus. The tissue may be a mucosal tissue that forms the innermost wall of the intestines. In other embodiments, the tissue may be a serosal tissue that forms the outermost wall of the intestines. As used herein, a “satiety hormone” is a factor secreted from endocrine tissue(s) that, via interaction with its receptor(s), leads to a feeling of satisfaction and/or fullness that results in appetite suppression, reduction in food intake, or both. An exemplary satiety hormone is GLP-1. “Stimulation of the release of satiety hormone” embraces both direct and indirect stimulation of release of hormone; for example, the electrical stimulus may be a direct cause of the release of hormone, such as from the L-cells, and/or the electrical stimulus may induce a cascade or series of events that ultimately results in the release of satiety hormone. Such cascade or series of events may include stimulation of one type of satiety hormone that in turn leads to the release of one or more additional types of satiety hormone or additional quantities of the first type of satiety hormone.

The first electrical stimulus may be applied to any tissue of the gastrointestinal system. For example, the stimulus may be applied to a mucosal tissue of the ileum; in particular instances, the stimulus may be applied to a mucosal tissue of the distal ileum. In contrast with existing methods, the present invention may include the application of electrical stimulus to a mucosal tissue lining the lumen of the gastrointestinal system, as opposed to exclusively applying an electrical stimulus to an outside surface of a gastrointestinal organ, such as to the serosa of the stomach or intestine. It has been discovered that direct stimulation of mucosal tissue in combination with the other specified aspects of this invention provides highly favorable results.

It has presently been discovered that the use of specific electrical parameters during the application of the first electrical stimulus are preferred for optimal release of satiety hormone. Exemplary electrical parameters that may be varied in accordance with the present invention include frequency, voltage, and pulse duration. The first electrical stimulus may have a frequency of about 0.1 Hz to about 90 Hz; for example, the stimulus may have a frequency of about 0.1 Hz, about 0.15 Hz, about 0.2 Hz, about 0.4 Hz, about 1 Hz, about 4 Hz, about 10 Hz, about 20 Hz, about 25 Hz, about 30 Hz, about 35 Hz, about 40 Hz, about 50 Hz, about 70 Hz, or about 90 Hz. The first electrical stimulus may have a voltage of about 0.5 V to about 25 V; for example, the voltage may be about 1 V, about 2 V, about 5 V, about 10 V, about 15 V, about 20 V, or about 25 V. In particularly preferred embodiments, the voltage is about 14 V. The first electrical stimulus may have a pulse duration of about 3 ms to about 500 ms; for example, the pulse duration may be about 5 ms, about 50 ms, about 100 ms, about 150 ms, about 200 ms, about 250 ms, about 300 ms, about 350 ms, about 400 ms, about 450 ms, or about 500 ms.

In some embodiments, the first electrical stimulus may be applied at a voltage of about 14V, with a pulse duration of about 5 ms, and at a stimulus frequency of about 20 to about 80 Hz; with respect to such embodiments, the stimulus frequency may be, for example, about 20 Hz, about 40 Hz, or about 80 Hz. In other aspects, the first electrical stimulus may be applied at a voltage of about 14 V, with a pulse duration of about 300 ms, and at a frequency of about 0.4 Hz.

The electrical stimulus that is applied to a tissue in the lumen of the gastrointestinal system of the subject may also be expressed in terms of charge, the unit for which is microCoulombs (μC), and otherwise referred to as “Q”. The first electrical stimulus may have a charge of greater than 3 μC. In other aspects, the first electrical stimulus may have a charge of between about 3 μC and about 6000 μC, inclusive. In a particular embodiment, the first electrical stimulus has a charge of about 1680 μC. Another embodiment involves the application of first electrical stimulus that has a charge of about 2800 μC. Other embodiments involve the application of a first electrical stimulus that has a charge of about 3.75 μC, about 7.5 μC, about 15 μC, about 31.5 μC about 280 μC, about 1400 μC, or about 5600 μC.

In accordance with the present invention, the first electrical stimulus is applied to a tissue in the lumen contemporaneously with the contacting of L-cells of the tissue with a nutrient stimulus. As used herein, “contemporaneously” means that during at least part of the time that the electrical stimulus is applied to the tissue, the L-cells are contacted with the nutrient stimulus. Thus, if the first electrical stimulus is applied for a total duration of one second, contacting the L-cells with the nutrient stimulus for 5 seconds after the application of the first electrical stimulus and for 0.1 seconds during the application of the first electrical stimulus will be considered to have been contemporaneous with the application of the first electrical stimulus. The contacting of the L-cells of the tissue with a nutrient stimulus refers to direct contact of the L-cells with the nutrient stimulus. This is to be contrasted with methods whereby electrical stimulation was timed to occur responsively to the mere act of eating (such as by generally sensing stomach physiological parameters indicative of ingestion, including interpreting electrical activity of the stomach, sensing antral contractions indicative of the onset or imminent onset of eating, detecting ectopic sites of natural gastric pacing, or sensing efferent neural modulation of gastric electrical activity) or the detection of generally elevated blood glucose levels (see, e.g., U.S. Pub. No. 2007/0179556 at paragraphs [0191]-[0223]).

The nutrient stimulus may comprise any substance that is capable of provoking a release of one or more hormones from L-cells. Exemplary nutrient stimulus substances include carbohydrates, other sugars, amino acids, proteins, fatty acids, fats, or any combination thereof. The nutrient stimulus may take the form of a natural food item, a supplement (such as a nutrition drink), or a substance that is made with the express purpose of stimulating L-cells, and therefore need not be a “nutrient” per se in the conventional sense.

In additional embodiments of the present invention, the first electrical stimulus may be applied to more than one location on the gastrointestinal tissue of the subject. For example, the first electrical stimulus may be applied to two, three, four, or more locations in the distal ileum of the subject. A “location” may be defined by the area of physical contact between the tissue and the means for delivery of the electrical stimulus (e.g., an electrode). Accordingly, the application of the first electrical stimulus to a second location on the gastrointestinal tissue of the subject may comprise contacting an electrode with a portion of the tissue that is not in physical contact with the means for delivery of the electrical stimulus to the original location on the gastrointestinal tissue.

The instant invention may further comprise applying a second electrical stimulus to the gastrointestinal tissue of said subject. The second electrical stimulus may be applied to the same location on the same gastrointestinal tissue as that to which the first electrical stimulus is applied, to a different location on the same gastrointestinal tissue, to a second tissue of the gastrointestinal system of the subject, or any combination thereof. The second electrical stimulus may be applied to a tissue of the duodenum (e.g., a mucosal tissue of the duodenum), a tissue of the jejunum (e.g., a mucosal tissue of the jejunum), or a tissue of the large intestine of said subject (e.g., a mucosal tissue of the large intestine); where the first electrical stimulus is applied to the distal ileum, for example, the second electrical stimulus may be said to have been applied to a second luminal tissue of the subject. The second electrical stimulus may differ from the first electrical stimulus in terms of voltage, frequency, pulse duration, charge, or any combination thereof.

The second electrical stimulus may be applied contemporaneously with the application of the first electrical stimulus. In this context, “contemporaneously” means that during at least part of the time that the first electrical stimulus is applied to a tissue, the second electrical stimulus is applied to a same or different location of that tissue, or to a different tissue, as the case may be. Thus, if the first electrical stimulus is applied for a total duration of one second, application of the second electrical stimulus for 5 seconds after the application of the first electrical stimulus and for 0.1 seconds during the application of the first electrical stimulus will be considered to have been contemporaneous with the application of the first electrical stimulus.

Electrical stimulation of tissue in accordance with the present invention can provide a benefit for patient diagnosis. There exists a need for a method of patient segmentation to determine the best candidates for surgical treatment for obesity. In accordance with the present invention, there are also provided methods for predicting patient response to a weight loss surgery comprising applying a first electrical stimulus to a tissue of the gastrointestinal system of said patient contemporaneously with the contacting of L-cells of the tissue with a nutrient stimulus; assessing the effect of the electrical stimulus in the patient; and, correlating said effect to the patient's response to a weight loss surgery.

As used herein, “weight loss surgery” includes bariatric surgery, implantation surgery, or any other surgical procedure that is intended to modify one or more parts of the gastrointestinal tract to reduce nutrient intake and/or absorption, to decrease appetite, or to induce weight loss and/or the maintenance of a desired body weight. Exemplary weight loss surgeries include, inter alia, biliopancreatic diversion, vertical banded gastroplasty, adjustable gastric banding, sleeve gastrectomy, gastric bypass surgery, sleeve gastrectomy with duodenal switch, and implantable gastric stimulation.

The application of the electrical stimulus may be performed in accordance with the preceding discussion with respect to the disclosed methods for stimulating the release of satiety hormone. In general, the definitions and parameters described with respect to the disclosed methods for stimulating release of satiety hormone are fully applicable to the present methods for predicting patient response to a weight loss surgery.

The assessment of the effect of the electrical stimulus in the patient may comprise a determination of the existence, and optionally the extent, of one or more physiological and/or psychological parameters associated with the ileal brake process, satiety, appetite modulation, or any combination thereof. For example, the assessment of the effect of the electrical stimulus may comprise a determination of the existence, extent, or both of blood levels of one or more satiety and/or ileal brake hormones, glucose or both, a feeling of fullness on the part of the patient, slowed gastric emptying and/or satiety in response to the nutrient stimulus, or any combination thereof. In particular examples, the assessment of the effect of the electrical stimulus may comprise a determination of the level of circulating GLP-1 in response to a test meal, improvement in glucose control (for example, as shown by such tests as Glucose Tolerance and Hba_{1c}), earlier perception of fullness and/or satisfaction (satiety) in response to a meal and earlier cessation of eating a meal, and the like. Commonly used Visual Analog Scales that could be applied to measure perception of appetite and satiety by manual or electronic recording include Three Factor Eating questionnaire; Appetite, Hunger and Sensory Perception questionnaire (AHSP); Council for Nutrition Appetite Questionnaire (CNAQ) and Simplified Nutrition Appetite Questionnaire (SNAQ) Appetite and Diet Assessment Tool (ADAT)

The assessed effect of the electrical stimulus in the patient may be correlated to an increased likelihood of a favorable patient response to therapeutic intervention, such as treatment with a drug that increases GLP-1 levels or weight loss surgery. For example, in instances wherein there is an enhancement of one or more physiological and/or psychological parameters associated with the ileal brake process, satiety, appetite modulation, or any combination thereof. A regression analysis of the extent by which the measures described in the preceding paragraph improved in response to localized electrical stimulation and actual improvements in weight loss and T2D in patients subsequently undergoing bariatric surgery would establish the predictability of the test as a means for patient stratification for bariatric surgery.

Accordingly, a minimally-invasive approach using electrical stimulation in accordance with the present methods may be used to predict patient response prior to treatment and would improve the likelihood of positive outcome. After endoscopic placement (preferably temporarily, but optionally permanently or over a long period of time) of an appropriate device at or near the site of stimulation, a patient would be monitored for enhancement of blood levels of ileal brake hormones or glucose, a feeling of fullness, slowed gastric emptying or satiety in response to a second nutrient stimulus, i.e., a nutrient stimulus that is distinct from the nutrient stimulus in accordance with the present methods, such as a nutrient meal, such as a nutrition drink or a standard caloric meal. This may be used to predict tangible therapeutic benefits of weight loss and improved glucose control that would improve the likelihood of a positive outcome after electing to undergo drug treatment and/or general anesthesia and bariatric or implantation surgery in obese and diabetic patients.

In accordance with any of the presently-disclosed methods, monitoring of the patient may also constitute an aspect of ongoing patient care and follow-up to allow adjustment and fine-tuning of the stimulation parameters over time. Thus, the present methods may include alteration of one or more parameters of the application of electrical stimulus, such as the first electrical stimulus, a second electrical stimulus, or both, over time. The alteration may occur with respect to two separate time points (for example, at t=1 a first stimulatory regime may be used, with a different stimulatory regime applied at t=2), or with respect to multiple time points. The alteration may involve the increase or decrease of one or more of such stimulatory parameters as frequency, voltage, pulse duration, charge, and location.

One objective of altering one or more stimulatory parameters may be the determination of optimal stimulatory conditions. For example, one or more preferred locations for the application of electrical stimulus may be determined in accordance with the present techniques. The determination of optimal stimulatory conditions may be performed with respect to a particular patient class (for example, male patients, female patients, patients grouped according to age, minimally obese patients, moderately obese patients, severely obese patients, patients of average weight with diabetes, obese patients without diabetes, obese patients with diabetes, and the like), or with respect to an individual patient.

In another aspect, the lowest optimal electrical stimulus parameter, e.g., frequency, voltage, pulse duration, and/or charge, that is associated with a subsequent positive stimulatory response may be determined A positive stimulatory response may include, for example, increased circulating GLP-1 levels in response to a test meal, improvement in glucose control as shown by routine tests (Glucose Tolerance and Hba_{1c}), earlier perception of fullness and/or satisfaction (satiety) in response to a meal and earlier cessation of eating a meal, and the like. Accordingly, a minimal electrical stimulus may be applied to a gastrointestinal tissue of a patient, and one or more parameters of the stimulus may be increased until at least one sufficient response is obtained and maintained at that level of stimulus.

### Example 1

### Measurement of GLP-1 Release From Isolated Small Intestine

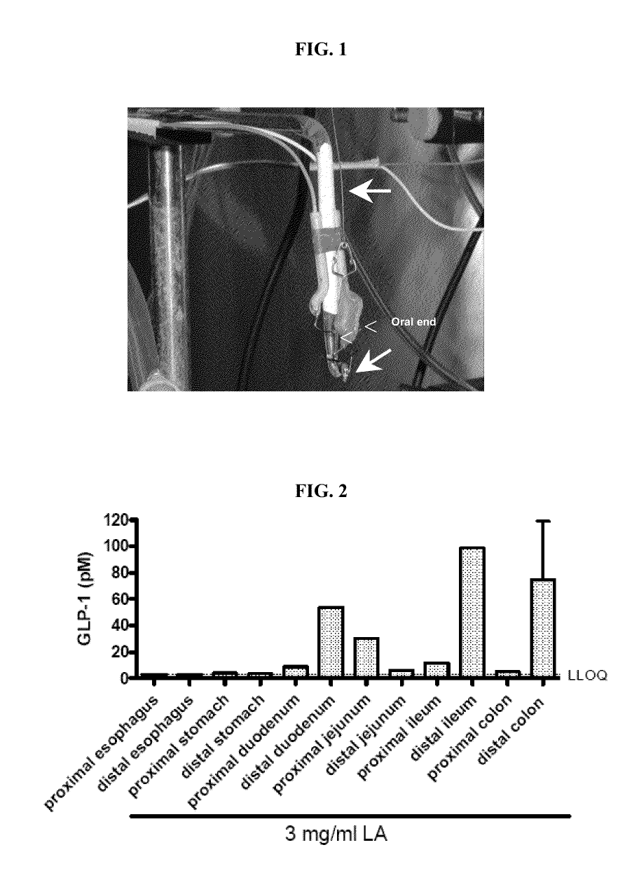

Female Sprague-Dawley rats, 8-12 weeks weighing 250-300 g were euthanized by CO₂ and at least 17 cm of distal ileum was immediately dissected starting at the ileocecal junction. Intraluminal contents were flushed with warm modified Krebs Ringers bicarbonate (KRB) buffer and intestines placed into 50 mL tubes containing oxygenated, cold KRB buffer. Intact segments (1.5 cm) of rat distal ileum were oriented longitudinally, with the oral end fixed in the organ chamber between bipolar stimulating electrodes and the aboral end attached to a solid-state force transducer and submerged in a 10 ml-chamber containing KRB at 37° C. and constantly aerated with 95% O₂/5% CO₂ (FIG. 1). The image in FIG. 1 shows the location of electrodes tips (arrow heads) relative to the ileum which is mounted with oral end closest to the electrode and held under tension between a glass hook and wire to force transducer (arrows). The entire assembly was placed into 37° C. KRB buffer in jacketed 10 mL myobath chambers. The length of each segment was adjusted to an initial resting tension of 1 g and maintained at 37° C. in a KRB buffer or KRB containing linoleic acid (LA, 3 mg/mL) and a dipeptidyl peptidase-4 inhibitor (to prevent proteolysis of GLP-1). Contractile activity was digitized and data acquired for off line analysis using PowerLab hardware and Chart software (ADInstruments, Colorado Springs, Colo.). In separate experiments, segments were incubated in KRB or KRB+LA in the presence or absence of electrical field stimulation continuously for 45 min. Samples of the bathing solutions taken at 45 min and mucosal epithelial scrapings were stored frozen (minus 80° C.).

Active GLP-1 concentration in thawed aliquots was measured by fluorescence on a plate reader using an ELISA (Linco Research, St. Charles, Mo.) with a detection range of 2-100 μM. This method measures both biologically active forms of GLP-1, that is GLP-1 (7-36) and (GLP-1(7-36)) amide, that are currently known to be released by the intestinal mucosa. Measurements of GLP-1 were normalized to concentration in 10 mL volume and reported as pM. The mean and SEM GLP-1 release was calculated for each treatment. For each electrical stimulation condition (+/−LA) there were 2-6 rats with 2-4 segments of tissue per rat per condition.

Muscle tone and contractile amplitude (calculated as the average cyclic minimum and maximum, respectively) were determined for 5 min periods, pre- (˜5 min before) and post-treatment (40 min after start). The tone and amplitude at −5 and +40 minutes for each condition was compared to that condition's baseline by one-way ANOVA.

Tissue incubated in 1, 3 and 10 mg/mL LA resulted in a maximal GLP-1 response at 3 mg/ml (data not shown), and this concentration was used for all subsequent experiments. GLP-1 concentration increased in the bathing medium, when segments of duodenum, jejunum ileum and colon, but not esophagus or stomach were incubated in LA (3 mg/mL) for 45 minutes (FIG. 2). FIG. 2 shows the concentration of GLP-1 in segments from the entire GI tract released after 45 min incubation in 3 mg/mL LA (LLOQ=lower limit of quantification).

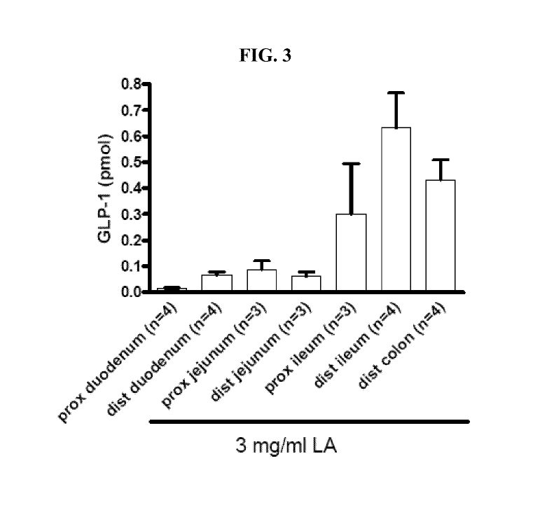

This regionally dependent release in isolated segments is consistent with the known location of L-cells in the intestines and their absence in the upper gastrointestinal tract, i.e., the stomach and esophagus. The mucosa from the small and large intestine was also analyzed for GLP-1 content (FIG. 3). FIG. 3 shows that GLP-1 is detectable in the epithelium of the duodenum, jejunum ileum and colon. The mucosal scrapings in small intestine and colon were sampled after 45 minutes incubation in LA for 45 mins (n=number of segments, Mean+SEM). The highest amount of GLP-1 in the mucosa was in the distal ileum (FIG. 3). Therefore, the distal ileum was selected for study of GLP-1 release for all subsequent experiments.

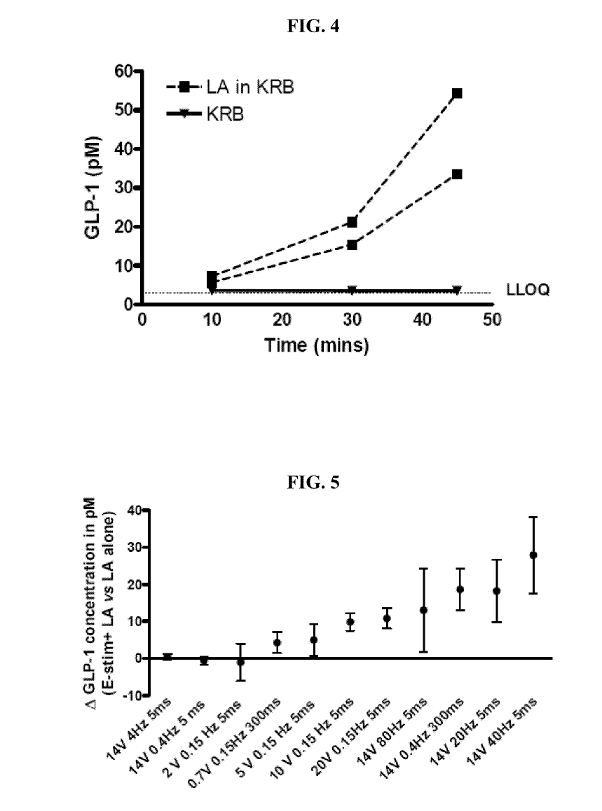

GLP-1 concentration from two segments of ileum incubated in 3 mg/ml LA increased over time, whereas GLP-1 concentration from ileal segments incubated in KRB buffer was at or below level of quantification (FIG. 4). Compiled data from 51 distal ileum segments showed that GLP-1 released after 45 mins incubation in LA was significantly greater (21.9±2.6 pM GLP-1) than after incubation in KRB buffer alone (3.6±0.1 pM GLP-1; P<0.05 by t-test; n=12).

### Example 2

### Measurement of GLP-1 Release Under Electrical Stimulation Conditions

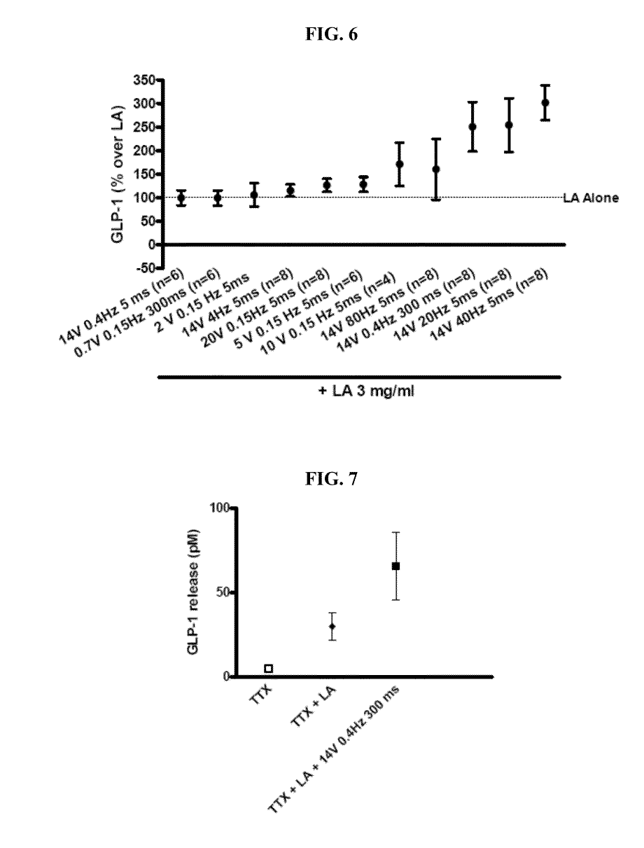

A total of eleven electrical stimulation conditions were selected for assessment. The results are shown as the difference in absolute change in GLP-1 concentration (FIG. 5), and as a percentage (FIG. 6), relative to control segments of ileum from the same rats incubated in LA. The data represented are consistent for seven electrical stimulation conditions. As provided in FIG. 6, eight of the conditions increased GLP-1 release over that expected when incubated in LA alone, normalized to 100%. As provided in FIG. 5, eight conditions resulted in an increase in the concentration of GLP-1. As provided in FIG. 5, 0.7 V 0.15 Hz 300 ms increased GLP-1 above the concentration in response to LA alone, and in FIG. 6 14V 4 Hz 5 ms increased the percentage of GLP-1 above LA normalized to 100%. As provided in FIGS. 5 and 6, two conditions did not increase GLP-1 above LA as shown by either analysis. These are 14V 0.4 Hz 5 ms and 2V 0.15 Hz 5 ms. Electrical stimulation conditions applied to the tissue in the absence of LA did not result in detectable amounts of GLP-1 release (2.3±0.2 μM GLP-1 n=46, with 38 of 46 samples below levels of detection by ELISA).

Effect of Neurotoxin on Stimulation of GLP-1 Release

To determine the effect of electrical stimulation via nerves in the tissue segments, tetrodotoxin (TTX), a commonly used toxin to block sodium channels in neurons, was added to the final KRB tissue wash for 15 min at a concentration of 0.5 μM (15 min preincubation). TTX was present at 0.5 μM concurrently with linoleic acid and/or electrical stimulation for 45 min. TTX alone had no effect on GLP-1 release, and LA-evoked increase GLP-1 persisted in the presence of TTX (FIG. 7). Thus, neuronal sodium channel activation is not required for LA to interact with its receptor on L-cells and evoke a release of GLP-1. Despite the presence of TTX, electrical stimulation (14V 0.4 Hz 300 ms) together with LA increased GLP-1 release by 239±64% over that evoked by LA alone. This is similar to the LA enhancement in GLP-1 evoked by the same electrical stimulation conditions in the absence of TTX (FIG. 6). From this it is concluded that neuronal activation is neither necessary nor sufficient for electrical stimulation to enhance the LA-evoked GLP-1 release from L-cells.

Statistical analysis was performed that included all conditions (with condition defined by the combination of frequency, voltage and duration of the electrical stimulation) and included a term for condition, treatment (LA alone or LA plus electrical stimulation) as a repeated factor, and the interaction between the two. Treatment is a within-subject effect, allowing each subject's LA alone response to serve as the control for that subject's response with electrical stimulation from the same study day. GLP-1 release was analyzed by repeated measures analyses of variance (ANOVA) on all eleven conditions with LA alone or LA plus electrical stimulation as a repeated factor. The mean and SEM reported in the tables below are based on 2-6 rats per condition with data from two replicates of each electrical stimulation condition averaged per rat. The P values are reported based on repeated measures analyses of variance (ANOVA) for each analysis and for the pairwise comparisons. The data were log-transformed prior to analyses to better satisfy the underlying statistical modeling assumptions of equal variability and sampling from populations with normal distributions.

The overall p-value for the effect of electrical stimulation when all eleven conditions are combined is p<0.001. Thus, it can be concluded that electrical stimulation plus LA significantly alters the amount of GLP-1 released compared to that released by LA alone. The Tables below summarize the individual P-values for each of the conditions, showing that by this stringent analysis two conditions resulted in a level of GLP-1 release which attained statistical level of significance.

Table 1, below, summarizes the results for five electrical stimulation conditions tested at 14V, 5 ms pulse duration with varying Hz. One of these conditions, 40 Hz, 14V and 5 ms results in a statistically significant difference in GLP-1 released compared to the tissue exposed to LA alone.

TABLE 1
Comparison of GLP-1 release in presence of LA alone versus LA plus
electrical stimulation at 14 volts and 5 ms with varying frequency

| | LA alone (pM) | LA + E-stim (pM) | |
|---|---|---|---|
| | (mean ± SEM) | (mean ± SEM) | p-value |
| 0.4 Hz 14 V 5 ms | 11.0 ± 2.8 | 10.4 ± 1.8 | 0.761 |
| 4 Hz 14 V 5 ms | 8.6 ± 1.8 | 9.1 ± 1.4 | 0.838 |
| 20 Hz 14 V 5 ms | 14.4 ± 5.9 | 32.6 ± 12.2 | 0.152 |
| 40 Hz 14 V 5 ms | 10.2 ± 2.1 | 38.1 ± 11.1 | 0.016 |
| 80 Hz 14 V 5 ms | 20.6 ± 3.4 | 33.7 ± 12.4* | 0.391 |

*Includes one result designated 100 pM which is the upper level of detection, although the value obtained was actually 178 pM.

Four electrical stimulation conditions were assessed wherein the frequency and pulse duration were kept constant at 0.15 Hz and 5 ms and the voltage was varied (Table 2, below). None of these conditions significantly increased GLP-1 release compared to LA alone.

TABLE 2
Comparison of GLP-1 release in presence of LA alone versus LA plus
electrical stimulation at 0.15 Hz and 5 ms duration with varying voltage

| | LA alone (pM) | LA + E-stim (pM) | |
|---|---|---|---|
| | (mean ± SEM) | (mean ± SEM) | p-value |
| 0.15 Hz 2 V 5 ms | 12.1 ± 4.0 | 11.1 ± 1.0 | 0.948 |
| 0.15 Hz 5 V 5 ms | 17.6 ± 5.9 | 22.6 ± 9.4 | 0.344 |
| 0.15 Hz 10 V 5 ms | 19.5 ± 9.0 | 29.4 ± 6.6 | 0.311 |
| 0.15 Hz 20 V 5 ms | 12.5 ± 1.7 | 16.5 ± 3.7 | 0.212 |

Next, two electrical stimulation conditions were applied which had a longer pulse duration of 300 ms and the results analyzed (Table 3, below). The increase in GLP-1 when incubated in LA plus 0.7 V, 0.4 Hz 300 ms duration approached statistical significance with this analysis (P=0.056). There was a small but consistent increase of electrical stimulation at 0.15 Hz, 0.7 V and 300 ms.

TABLE 3
Comparison of GLP-1 release in presence of LA alone versus LA plus
electrical stimulation at two conditions with pulse duration of 300 ms

| | LA alone (pM) | LA + E-stim (pM) | p- |
|---|---|---|---|
| | (mean ± SEM) | (mean ± SEM) | value** |
| 0.15 Hz 0.7 V 300 ms | 17.9 ± 4.1 | 22.1 ± 4.2 | 0.257 |
| 0.4 Hz 14 V 300 ms | 12.7 ± 2.4 | 31.3 ± 5.9 | 0.056 |

From the preceding analysis it was possible to identify two conditions where the results are unlikely to be due to chance and these are 40 Hz, 14 V, 5 ms and 0.4 Hz, 14V, 300 ms. Additionally, when the conditions are compared with each other, statistically significant differences between the conditions and the amount of GLP-1 release (p=0.029) become apparent.

Taking into account this statistical analysis and the compiled responses (FIGS. 5 & 6) it was shown that all but two electrical stimulation conditions enhanced the amount of GLP-1 released during incubation with a nutrient stimulus The two conditions that had no apparent effect on the amount of GLP-1 release were 14 V 0.4 Hz 5 ms, 14 V 4 Hz 5 ms, 2 V 0.15 Hz 5 ms). The remaining nine electrical stimulation conditions increased GLP-1 levels above that induced by LA by varying degrees.

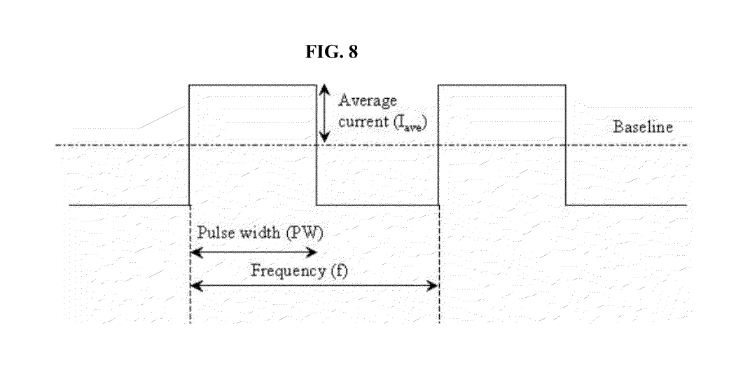

Another way of analyzing these data is to estimate the approximate electrical charge of the eleven electrical stimulation conditions. The resulting ‘Q’ is a product of current and time and relates to the “electrical charge” delivered during stimulation. In an electrical stimulation application, the charge delivered through electrodes or contact surfaces serves as a measure of efficacy. The result can be expressed in charge per phase or charge per unit area. Total charge delivered is defined as the product of current and the duration for which it is delivered. FIG. 8 illustrates that the average charge (Qₐᵥₑ) delivered per phase during stimulation is a function of the average current (Iₐᵥₑ) and pulse width (PW).

Qₐᵥₑ=Iₐᵥₑ*t

In the absence of a current waveform, charge delivered is obtained from the voltage applied and the impedance (Z) or resistance (R) as follows:

$${Q}_{ave} = \frac{{V}_{ave}}{ZorR} * t$$

Table 4, below, provides a comparison of Q (microCoulombs) for each electrical stimulation condition.

TABLE 4

| Voltage (V) | f (Hz) | PW (ms) | Q (μC) |
|---|---|---|---|
| 2 | 0.15 | 5 | 1.5 |
| 5 | 0.15 | 5 | 3.8 |
| 10 | 0.15 | 5 | 7.5 |
| 20 | 0.15 | 5 | 15 |
| 14 | 0.4 | 5 | 28 |
| 14 | 4 | 5 | 280 |
| 14 | 20 | 5 | 1400 |
| 14 | 40 | 5 | 2800 |
| 14 | 80 | 5 | 5600 |
| 0.7 | 0.15 | 300 | 31.5 |
| 14 | 0.4 | 300 | 1680 |

In general, the magnitude of the responses was correlated with Q for the eleven electrical stimulation conditions. The two conditions that had no apparent effect on the amount of GLP-1 release (14 V 0.4 Hz 5 ms, 2 V 0.15 Hz 5 ms) had a total charge of 28 μC and 1.5 μC, respectively. An increase was noted when with total charge of 3.8 μC. The four conditions that increased GLP-1 release 150-300% had total charges of 1400, 1680, 2800 and 5600 μC. However, for a total charge <100 μC the extent of electrical stimulation enhancement of GLP-1 can be optimized by differing the combinations of frequency, pulse width, and voltage strength.

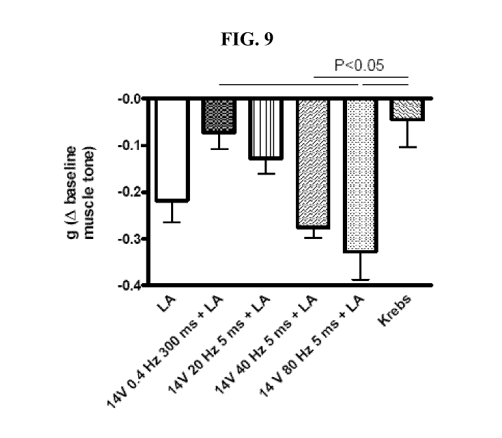

Contractility and tone were recorded in addition to GLP-1 release. Incubation in LA alone tended to decrease tone of the isolated ileum after 40 min although only when 14V 40 Hz 5 ms and 14 V 80 Hz 5 ms was combined with LA was there a significant reduction in tone compared to incubation in KRB buffer (FIG. 9; P<0.05 by ANOVA). Muscle tone after incubation in LA with electrical stimulation parameters of 14V 0.4 Hz and 300 ms and 14V 20 Hz and 5 ms was not different than in KRB buffer alone.

In conclusion, GLP-1 release and smooth muscle contractile activity were measured in the isolated intestinal tissue segments in the presence of LA and eleven electrical stimulation conditions. In general, the magnitude of the responses was correlated with the total charge. Four electrical stimulation conditions enhanced the amount of GLP-1 released during incubation with a nutrient stimulus by 150-300% compared to LA alone and these had total charge level >1400 μC. Two of these conditions were not associated with significant changes in smooth muscle tone (14V 0.4 Hz 300 ms and 14V 20 Hz 5 ms). Two conditions (14 V 80 Hz 5 ms and 14 V 40 Hz 5 ms) were associated with a decrease in muscle tone that was similar to the effect of LA alone. Without intending to be bound by any particular theory, this suggests that the effects of electrical stimulation on hormone release may be independent of effects on smooth muscle. When the total charge is <100 μC the extent of electrical stimulation enhancement of GLP-1 can be optimized by differing the combinations of frequency, pulse width, and voltage strength. From this it is concluded that there are specific electrical energy requirements for enhancing GLP-1 release locally in the small intestine that are dependent for their effects on the presence of a fatty acid stimulus.

Thus, electrical stimulation of the small intestine that can favorably change the release of a suite of hormones from endocrine cells in response to a natural (food) stimulus would provide a power-assist to the ileal brake. This would be expected to reduce weight in obese patients and increase insulin release and glucose utilization for improved glycemic control in T2D patients. It may also be used as a temporarily placed device through a natural orifice in the lumen of the intestines and combined with nutrient stimulation for detection of enhanced circulating hormone release (e.g., GLP-1) and patient reported sensations of fullness. This diagnostic would identify patients most likely to benefit therapeutically from surgical and permanent treatment with an electrical device for improved weight control and diabetes. It could also be used to optimize the location or delivery of electrical stimulus and duration to achieve beneficial feeling of fullness and glycemic control, while minimizing adverse effects.

1. A method of stimulating the release of satiety hormone in a subject comprising: applying a first electrical stimulus to a tissue of the gastrointestinal system of said subject contemporaneously with a contacting of L-cells of the tissue with a nutrient stimulus.

2. The method according to claim 1 wherein the first electrical stimulus is applied to a mucosal tissue of the gastrointestinal system of the subject.

3. The method according to claim 1 wherein said first electrical stimulus is applied to a mucosal tissue of the ileum.

4. The method according to claim 3 wherein said first electrical stimulus is applied to a mucosal tissue of the distal ileum.

5. The method according to claim 1 wherein the first electrical stimulus is applied at a frequency of about 0.1 Hz to about 90 Hz.

6. The method according to claim 1 wherein the first electrical stimulus is applied at a voltage of about 0.5 V to about 25 V.

7. The method according to claim 1 wherein the first electrical stimulus has a pulse duration of about 3 ms to about 500 ms.

8. The method according to claim 1 wherein the first electrical stimulus is applied at a voltage of about 14V, with a pulse duration of about 5 ms, and at a frequency of about 20 to about 80 Hz.

9. The method according to claim 8 wherein the first electrical stimulus is applied at a frequency of about 40 Hz.

10. The method according to claim 1 wherein the first electrical stimulus is applied at a voltage of about 14 V, with a pulse duration of about 300 ms, and at a frequency of about 0.4 Hz.

11. The method according to claim 1 wherein the first electrical current has a charge of greater than 3 μC.

12. The method according to claim 1 wherein the first electrical current has a charge of about 3 μC to about 6000 μC, inclusive

13. The method according to claim 1 comprising applying said first electrical stimulus to more than one location on the luminal tissue of said subject.

14. The method according to claim 1 further comprising applying a second electrical stimulus to the luminal tissue of said subject.

15. The method according to claim 14 wherein said second electrical stimulus differs from the first electrical stimulus in terms of voltage, frequency, pulse duration, charge, or any combination thereof.

16. The method according to claim 1 further comprising applying a second electrical stimulus to a second tissue in the lumen of the gastrointestinal system of said subject at a location that differs from that to which said first electrical stimulus is applied.

17. The method according to claim 16 wherein said first electrical stimulus is applied to the ileum of said subject, and wherein said second electrical stimulus is applied to a luminal tissue of the duodenum, a luminal tissue of the jejunum, or a luminal tissue of the large intestine of said subject.

18. The method according to claim 16 wherein said second electrical stimulus is applied contemporaneously with the application of said first electrical stimulus.

19. The method according to claim 16 wherein said second electrical stimulus differs from the first electrical stimulus in terms of voltage, frequency, pulse duration, charge, or any combination thereof.

20. The method according to claim 1 wherein said nutrient stimulus comprises a carbohydrate, amino acid, proteins, fatty acid, fat, a substance made with the express purpose of stimulating L-cells, or any combination thereof.

21. The method according to claim 1 wherein said satiety hormone comprises Glucagon-Like Peptide-1 (GLP-1).

22. A method for predicting patient response to a weight loss surgery comprising: applying a first electrical stimulus to a tissue of the gastrointestinal system of said patient contemporaneously with a contacting of L-cells of the tissue with a nutrient stimulus; assessing the effect of the electrical stimulus in said patient; and, correlating said effect to said patient's response to said weight loss surgery.

23. The method according to claim 22 wherein said assessing comprises: determining the level of one or more satiety hormones, one or more ileal brake hormones, glucose, or any combination thereof in the blood of said patient; assessing the existence, enhancement, or both of a feeling of fullness on the part of said patient; assessing the existence, enhancement, or both of gastric emptying, satiety, or both in response to a second nutrient stimulus in said patient; or any combination thereof.

24. The method according to claim 23 wherein said assessing comprises determining the level of circulating GLP-1 in the blood of said patient.
